(19) United States
(12) Patent Application Publication
(10) Pub. No.: US 2012/0065565 A1
(43) Pub. Date: Mar. 15, 2012

(54) Tender Skin Bandage

(76) Inventor: Ervin Ray Tart, Midlothian, TX (US)

(21) Appl. No.: 12/878,942

(22) Filed: Sep. 9, 2010

(51) Int. Cl. A61F 13/02

(52) U.S. Cl. 602/43

(57) ABSTRACT

A disposable wrap for treating a wound without tearing tender skin. The wrap is designed with an opening so that a band aid can be placed on top of it the opening so that the adhesive will not touch the skin. The soft wrap is fastened to fit with Velcro. When bandage needs changing, this complete wrap can be thrown away. This will keep the elderly patients skin from being torn with the removal of the band aid as the adhesive will never touch the skin. The wrap and band aid will be packaged together for convenience.

### BACKGROUND OF THE INVENTION

The present invention relates to a wound dressing. It is disposable with the ability to use a band aid without the adhesive touching the skin. This keeps the skin from being torn when the band aid is removed.

### BRIEF SUMMARY OF THE INVENTION

This bandage to protect the tender skin of patients will help in the healing process by not causing more damage to the skin when the bandage is removed.

### BRIEF DESCRIPTION OF THE SEVERAL VIEWS OF THE DRAWING

FIG. 1 Shows the placement of the bandage on the arm. It easily adjusts to fit to any size arm or leg.

FIG. 2 Is a view of the top of the bandage. The band-aid will be placed over this opening not allowing the adhesive to touch the skin.

FIG. 3 Is a view of the bottom of the bandage. The Velcro adheres to the fabric to fasten bandage around wound.

FIG. 4 Is a view of the top of the bandage with the band-aid in place.

FIG. 5 Is a drawing of the band-aid that is used to place over the opening in the top of the bandage.

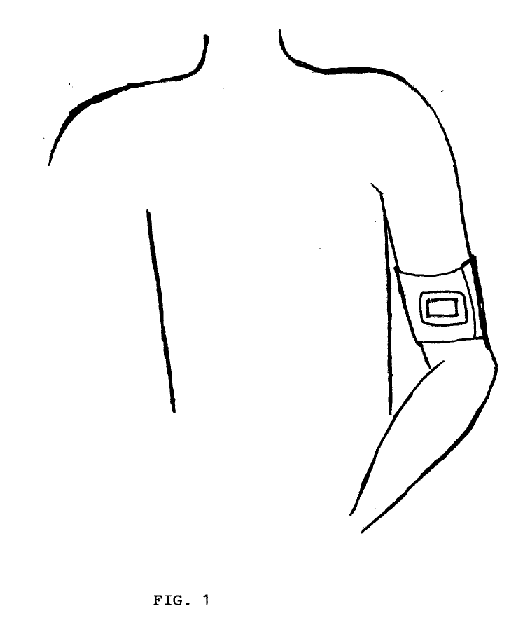

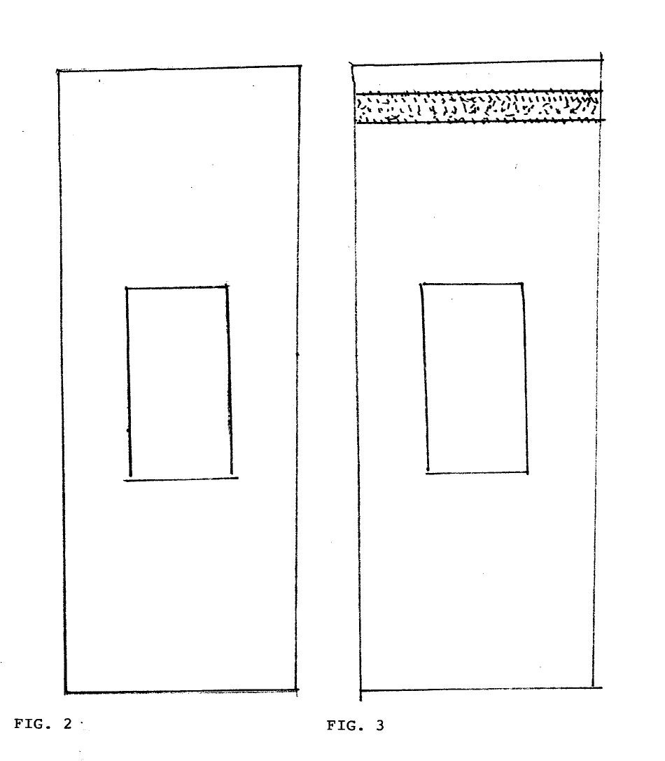

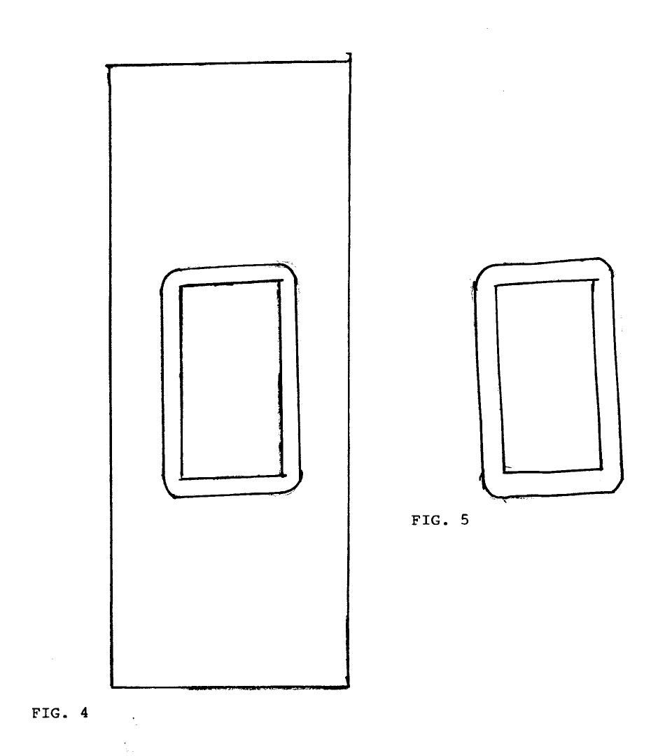

### DETAILED DESCRIPTION OF THE INVENTION

This Tender Skin Bandage is made of a soft fabric in a rectangular shape. It is Disposable and has a rectangular opening that will allow a sterile band-aid to be placed over it. The sterile band-aid is placed on the fabric over the opening so that the adhesive only touches the fabric of the bandage and not the patients skin. Velcro is attached to one end of the fabric so it will easily adjust to different sizes of patients arms or legs.

In the past, patients with tender and sensitive skin have had to suffer with tape or adhesive band-aids being stuck to their skin when being treated for open wounds or having an IV secured, etc. With the opening in the bandage wrap, it can be used many different ways to treat patients needs. The use of adhesive and tape on the patients skin causes more tears in the skin when it is removed and causes more pain and sores to be healed. This “Tender Skin Bandage will provide a comfortable and painless way to dress and heal wounds. The “Tender Skin Bandage” will be packaged with a sterile band-aid.

1. This invention will help patients heal without having additional tears to their skin.

2. In reference to claim number 1, the adhesive of the sterile band-aid does not touch the patients skin.

3. In reference to claim number 2, Now and in the past, patients have had much pain and have suffered from their skin being torn when adhesive band-aids or tape was put on their sensitive and tender skin for treatment and then removed.

4. In reference to claim number 3, This bandage will help the patients heal faster without suffering pain from the adhesive and tape used to adhere bandages.
